(19) United States
(12) Patent Application Publication
(10) Pub. No.: US 2025/0121346 A1
(43) Pub. Date: Apr. 17, 2025

(54) PREPARATION OF A NANOCOMPOSITE

(71) Applicant: APROXI APS, Herlev (DK)

(72) Inventors: Henrik Tofte JESPERSEN, Kirke Hyllinge (DK); Per Feifer BAEKGARD, Kirke Hyllinge (DK)

(21) Appl. No.: 18/517,648

(22) PCT Filed: May 23, 2022
(86) PCT No.: PCT/EP2022/063892

§ 371 Date: Nov. 22, 2023

(30) Foreign Application Priority Data
May 25, 2021 (DK) PA202100547

(51) Int. Cl. B01J 13/00; A61K 6/76; B82Y 40/00; C09D 7/61

(52) U.S. Cl. CPC B01J 13/0091; A61K 6/76; B82Y 40/00; C09D 7/61

(57) ABSTRACT

The present invention relates to a process for providing a nanocomposite, the process comprising the steps of (i) mixing a silica compound with one or more metal compounds in an aqueous solution providing a homogenous mixture; (ii) adjusting the pH of the homogenous mixture providing a pH-adjusted homogenous mixture; (iii) allowing pH-adjusted homogenous mixture silica to undergo a gelation process resulting in a hydrogel comprising an aqueous phase and an porous solid structure; (iv) Drying the hydrogel at a temperature and pressure combination that avoids boiling of the aqueous phase, providing the nanocomposite; wherein the nanocomposite provided may have a porous monolithic inorganic gel structure comprising a silica compound and one or more metal compounds.

### TECHNICAL FIELD OF THE INVENTION

The present invention relates to a process of providing a nanocomposite and a nanocomposite as such. In particular the present invention relates to a process for providing a nanocomposite comprising an porous solid structure having a porous monolithic inorganic gel structure comprising a silica compound and one or more metal compounds, and the nanocomposite as such.

### BACKGROUND OF THE INVENTION

Aerogels are synthetic porous ultralight materials derived from a gel, in which the liquid component for the gel has been replaced with a gas without significant collapse or shrinkage of the gel structure. The result is a solid with extremely low density and extremely low thermal conductivity.

Aerogels are solids with a porous, sponge-like structure in which about 95% of the volume is empty space (that is, filled with air). Aerogels are therefore unique among solid materials. They possess extremely low densities, large open pores, and a large inner surface area.

Aerogels are generally produced by extracting the liquid component of a gel structure through supercritical drying or freeze-drying. This allows the liquid to be slowly dried off without causing the solid matrix in the gel to collapse from capillary action, as would happen with conventional evaporation.

When a substance in a liquid body crosses the boundary from liquid to gas, the liquid changes into gas at a finite rate, while the amount of liquid decreases. When this happens within a heterogeneous environment, surface tension in the liquid body pulls against any solid structures the liquid might be in contact with. Delicate structures such as the dendrites in silica gel tend to be broken by this surface tension as the liquid-gas-solid junction moves by.

To avoid this, the substance to be removed can be brought from the liquid phase to the gas phase via two possible alternate paths without crossing the liquid-gas boundary.
freeze-drying, this means low temperature and low pressure. However, some structures are disrupted even by the solid-gas boundary.
Supercritical drying, applies high-temperature, high-pressure. This route from liquid to gas does not cross any phase boundary, instead passing through the supercritical region, where the distinction between gas and liquid ceases to apply. Densities of the liquid phase and vapor phase become equal at the critical point of the drying.

Fluids suitable for supercritical drying include carbon dioxide, freon, or nitrous oxide. However, supercritical water is considered unsuitable due to possible heat damage to a sample at its critical point temperature and corrosiveness of water at such high temperatures and pressures.

In most such supercritical drying processes, acetone is first used to wash away all water, exploiting the complete miscibility of these two fluids. The acetone is then washed away with high pressure liquid carbon dioxide, the industry standard now that freon is unavailable. The liquid carbon dioxide is then heated until its temperature goes beyond the critical point, at which time the pressure can be gradually released, allowing the gas to escape and leaving a dried product.

Hence, freeze drying and supercritical drying of the aqueous phase in the aerogel is undesirable because it involves a very complex processes with a lot of process steps which may influence the costs, added waste, use of additional process equipment's etc. which may be undesirable.

Hence, an improved method for providing a nanocomposite comprising an porous solid structure having a porous monolithic inorganic gel structure would be advantageous, and in particular a more efficient, simple, cost effective, environmental and/or reliable method for providing porous solid structures would be advantageous.

### SUMMARY OF THE INVENTION

Thus, an object of the present invention relates to a process of providing a nanocomposite comprising a porous solid structure having a porous monolithic inorganic gel structure and a nanocomposite comprising a porous solid structure having a porous monolithic inorganic gel structure as such.

In particular, it is an object of the present invention to provide a process for providing a nanocomposite comprising a porous solid structure having a porous monolithic inorganic gel structure wherein the porous solid structure comprising silica and one or more metal compounds, that solves the above-mentioned problems of the prior art with efficiency, simplicity, cost, environmental impact and/or reliability.

Thus, one aspect of the invention relates to a process for providing a nanocomposite, the process comprising the steps of:
(i) Mixing a silica compound with one or more metal compounds in an aqueous solution providing a homogenous mixture;
(ii) Adjusting the pH of the homogenous mixture providing a pH-adjusted homogenous mixture;
(iii) Allowing pH-adjusted homogenous mixture silica to undergo a gelation process resulting in a hydrogel comprising an aqueous phase and a porous solid structure;
(iv) Drying the hydrogel at a temperature and pressure combination that avoids boiling of the aqueous phase, providing the nanocomposite;

wherein the nanocomposite provided may have a porous monolithic inorganic gel structure comprising a silica compound and one or more metal compounds.

Another aspect of the present invention relates to a nanocomposite comprising an porous solid structure having a porous monolithic inorganic gel structure comprising a silica compound and one or more metal compounds obtainable by the method according to the present invention.

Yet another aspect of the present invention relates to a nanocomposite comprising an porous solid structure having a porous monolithic inorganic gel structure comprising a silica compound and one or more metal compounds, wherein the porous solid structure comprises an internal porous volume in the range of 40-90%, such as in the range of 50-80%, e.g. in the range of 60-75%, such as about 70%.

Still another aspect of the present invention relates to the use of an porous solid structure according to the present invention or a nanoparticulate material according to the present invention, as a filler in paint (preferably, in outdoor paints or marine paints; more preferably in outdoor paints); as a coating of a medical device; or as a dental filling.

Yet an aspect of the present invention relates to a paint (preferably, an outdoor paint or a marine paint; more preferably an outdoor paint); a medical device; or a dental filling, comprising a nanocomposite according to the present invention.

The present invention will now be described in more detail in the following.

### DETAILED DESCRIPTION OF THE INVENTION

Accordingly, the inventors of the present invention surprisingly found a process for providing a nanocomposite comprising a porous solid structure having a porous monolithic inorganic gel structure comprising a silica compound and one or more metal compounds, which process is more efficient, simpler, more cost effective, more environmentally friendly and/or more reliable and at the same time improving the properties of the porous solid structure obtained.

A preferred embodiment of the present invention relates to a nanocomposite comprising a porous solid structure having a porous monolithic inorganic gel structure comprising a silica compound and one or more metal compounds, wherein the porous solid structure comprises an internal porous volume in the range of 40-90%, such as in the range of 50-80%, e.g. in the range of 60-75%, such as about 70%.

The silica compound may be SiO₂ or a precursor thereof.

The nanocomposite according to the present invention may comprise an porous solid structure having a porous monolithic inorganic gel structure comprising an internal material.

The monolithic inorganic gel structure according to the present invention may comprise a mix of interconnected nanoparticulate silica compounds and one or more nanoparticulate metal compounds, preferably metal oxides (other than silica), all hold together by covalent (chemical) bonds.

In an embodiment of the present invention the monolithic inorganic gel structure may be a shaped, fabricated, intractable porous sold structure with a homogeneous microstructure which exhibit substantially no structural components distinguishable by optical microscopy

The porous solid structure may be formed by the porous monolithic inorganic (gel) structure creating an internal porous volume which may be highly interconnected and the internal porous volume may be filled with an internal material.

The internal material may be a gas, a liquid material, or a solid material. When the internal material may be a solid the solid material may be introduced as a liquid which is subsequently solidified inside the internal porous volume.

The internal material may or may not be covalently linked to the porous solid structure of the nanocomposite.

In an embodiment of the present invention the internal material may be a solid material and the solid material may be covalently linked to the porous solid structure of the nanocomposite.

In yet an embodiment of the present invention, the internal material is a gas, a liquid material, or a solid material; and the internal material may not be covalently linked to the porous solid structure of the nanocomposite.

In an embodiment of the present invention the nanocomposite consists essentially of an porous solid structure having a porous monolithic inorganic gel structure comprising a silica compound and one or more metal compounds according to the present invention.

In the context of the present invention, the term “consisting essentially of”, relates to a limitation of the scope of a claim to the specified features or steps and those features or steps, not mentioned and that do not materially affect the basic and novel characteristic(s) of the claimed invention.

In the context of the present invention, the terms “comprising”, may be used synonymously with “including”, “containing”, or “characterized by”, and are considered inclusive and open-ended terms which does not exclude additional, unrecited elements or process steps.

In an embodiment of the present invention the nanoparticulate material consists of a nanocomposite comprising a porous solid structure having a porous monolithic inorganic gel structure comprising a silica compound and one or more metal compounds according to the present invention

In the context of the present invention, the terms “consisting of” or “consist of” are considered closed-ended definitions and excludes any element, step, or ingredient not specifically mentioned after that phrase.

A nanoparticle may be characterized as an ultrafine particle and is in the present context defined as a particle of matter that is between 1 and 1000 nanometres (nm) in diameter.

Nanoparticles according to the present invention may be distinguished from microparticles, since microparticles may have a particle size above 1 μm, such as in the range of 1-1000 μm.

Since the particle structure of nanoparticles are only in theory considered ball-like shaped the particle size, or the average particle size, d50, and the particle size, d90, based on assumptions of the particle shape. Therefore, the particle size may be determined according to the specific surface area per gram particles.

In an embodiment of the present invention the specific surface area (a BET surface area) of the porous solid structure according to the present invention may be above 10 m²/g, such as above 25 m²/g, e.g. above 50 m²/g, such as above 75 m²/g, e.g. above 100 m²/g; such as above 150 m²/g.

In yet an embodiment of the present invention the porous solid structure according to the present invention may comprise a surface area (a BET surface area) below 1500 m²/g, such as below 1200 m²/g, e.g. below 1000 m²/g, such as below 800 m²/g, e.g. below 600 m²/g; such as below 500 m²/g, e.g. below 400 m²/g; such as below 350 m²/g, e.g. below 300 m²/g; such as below 200 m²/g.

The BET surface area (Brunauer-Emmett-Teller) is a theory that aims to explain the physical adsorption of gas molecules on a solid surface and serves as the basis for a conventional and important analysis technique for the measurement of the specific surface area of materials.

In an embodiment of the present invention the porous solid structure comprises a surface area (a BET surface area) in the range of 10-1500 m²/g, such as in the range of 20-1200 m²/g, e.g. in the range of 25-1000 m²/g, such as in the range of 30-800 m²/g, e.g. in the range of 40-600 m²/g; such as in the range of 50-300 m²/g, e.g. in the range of 75-250 m²/g; such as in the range of 100-200 m²/g, e.g. in the range of 150-500 m²/g; such as in the range of 200-400 m²/g, e.g. in the range of 300-350 m²/g.

The specific surface area may be determined according to the BET (Brunauer, Emmett and Teller), e.g. as described in ISO 9277:2010.

A preferred embodiment of the present invention relates to a nanocomposite comprising an porous solid structure having a porous monolithic inorganic gel structure comprising a silica compound and one or more metal compounds.

The porous solid structure provided according to the present invention may preferably comprise an internal porous volume in the range of 40-90%, such as in the range of 50-80%, e.g. in the range of 60-75%, such as about 70%.

The porous solid structure may comprise the one or more metal compounds dispersed in the silica compound.

In an embodiment of the present invention the one or more metal compound (provided in step (i)) may be one or more metal oxide compounds and/or one or more metal hydroxide compounds. Preferably, the one or more metal compound may be one or more metal oxide compounds.

Preferably, the metal compound may comprise a divalent metal ion, a trivalent metal ion, a tetravalent metal ion or a combination hereof. More preferably, the metal compound may comprise a trivalent metal ion, a tetravalent metal ion or a combination hereof.

The metal compound of the present invention does preferably not only include a univalent metal ion. Univalent metal ion may relate to a metal ion whose atoms is each capable of chemically combining with only one atom. This may not be preferred since the univalent metal ion in not capable of forming a covalent coupling between the molecules and thus not capable of forming a monolithic inorganic gel structure. Univalent metal ions may be used in small concentrations, in combination with one or more polyvalent metal ions. In the event the univalent metal ions may be used in a combination with one or more polyvalent metal ions the one or more polyvalent metal ions may be provided in highest concentration. Preferably the content of the univalent metal ions (if present) may be less than 1%, such as less than 0.5%, e.g. less than 0.1%. The univalent metal ions may preferably be Ag (silver ions).

A divalent metal ion may be a metal ion whose atoms are each capable of chemically combining with two atoms.

A trivalent metal ion may be a metal ion whose atoms are each capable of chemically combining with three atoms.

A tetravalent metal ion may be a metal ion whose atoms are each capable of chemically combining with four atoms.

The one or more metal compounds according to the present invention may be provided as:
(i) dispersed metal-oxide particles together with the silica compound;
(ii) dissolved metal salt, acting as a precursor for one or more metal-oxide compounds; or
(iii) a combination of dispersed metal-oxide particles together with the silica compound and dissolved metal salt, acting as a precursor for one or more metal-oxide compounds.

When the one or more metal compounds according to the present invention may be provided as a combination of dispersed metal-oxide particles together with the silica compound and one or more metal salt, acting as a precursor for the one or more metal-oxide compounds the one or more metal-oxide compounds may preferably comprise two or more metal-oxide compounds, such as having TiO₂ dispersed together with the silica compound and having ZrO₂ provided as a Zr-salt (such as Zirconyl chloride octahydrate or Zirconium (IV) oxynitrate hydrate) precursor which subsequently results in the formation of the ZrO₂.

Without being bound by theory it is believed that the metal compound (either added as dispersed metal-oxide particles or provided from a precursor for the one or more metal-oxide compounds, preferably provided from a precursor for the one or more metal-oxide compounds) and the silica compounds are covalently bound together by one or more oxygen bindings.

In an embodiment of the present invention the metal-oxide compound may be selected from a titanium oxide, a zirconium oxide, an aluminium oxide, a zinc oxide, magnesium oxide (MgO), silicon dioxide (SiO₂), or a combination hereof.

Preferably the titanium oxide may be titanium dioxide (TiO₂). Even more preferably, the titanium dioxide (TiO₂) may be in the crystalline form of anatase TiO₂ or rutile TiO₂, or a combination hereof. The crystalline form anatase TiO₂ being the preferred.

The zirconium oxide may preferably be zirconium dioxide (ZrO₂).

The aluminium oxide may be alumina (Al₂O₃).

The zinc oxide may be zinc dioxide (ZnO₂).

Metal-oxide nanoparticles have been extensively analysed for their physiochemical properties in biological applications and antimicrobial effects. I particular, TiO₂ has shown to be a strong antimicrobial agent, and are considered being a highly relevant additive in the present nanocomposite since it shows special features, such as easy control, reduced cost, non-toxicity, and good resistance to chemical erosion, that allow its application in various fields, but in particular as antibacterial and antifungal agents. In general, metal-oxide, in particular TiO₂ nanoparticles present large surface area, excellent surface morphology, and non-toxicity in nature.

The metal compound provided in step (i) may be provided as metal-oxide particles or as a salt of the metal oxide.

The particles may have a mean particle size (d50) below 50 nm, such as below 40 nm, e.g. below 30 nm, such as below 20 nm, e.g. below 10 nm, such as below 8 nm, e.g. below 6 nm, such as about 5 nm.

The particles may have a mean particle size (d50) above 1 nm, such as above 2 nm, e.g. above 3 nm, such as above 4 nm.

In an embodiment of the present invention the metal compound may have a mean particle size (d50) in the range of 1-50 nm, such as in the range of 2-20 nm, e.g. in the range of 3-10 nm, such as in the range of 4-6 nm.

The particles may have a specific surface area in the range of 50-250 m²/g, such as in the range of 75-225 m²/g, e.g. in the range of 100-200 m²/g, such as in the range of 125-175 m²/g, e.g. in the range of 150-165 m²/g.

In an embodiment of the present invention the metal compound may have a specific surface area above 75 m²/g, such as above 100 m²/g, e.g. above 125 m²/g, such as above 150 m²/g, e.g. above 160 m²/g, such as above 170 m²/g, e.g. above 200 m²/g.

The porous solid structure of the present invention may comprise a content of metal-compound in the range of 1-50 wt % of the porous solid structure (comprising silica compound and one or more metal compounds), such as in the range of 5-40 wt %, e.g. in the range of 10-50 wt %, such as in the range of 15-40 wt %, e.g. in the range of 20-30 wt %, such as about 25%.

In an embodiment of the present invention, the metal compound and/or the porous solid structure does not comprise an iron compound.

A preferred embodiment of the present invention relates to a process for providing a nanocomposite, the process comprising the steps of:
(i) Mixing a silica compound with one or more metal compounds in an aqueous solution providing a homogenous mixture;
(ii) Adjusting the pH of the homogenous mixture providing a pH-adjusted homogenous mixture;
(iii) Allowing pH-adjusted homogenous mixture silica to undergo a gelation process resulting in a hydrogel comprising an aqueous phase and a porous solid structure;
(iv) Drying the hydrogel at a temperature and pressure combination that avoids boiling of the aqueous phase, providing the nanocomposite;

wherein the nanocomposite provided may have a porous monolithic inorganic gel structure comprising a silica compound and one or more metal compounds.

The inventors of the present invention found that the monolithic inorganic gel structure may be preferred because it among other things provides improved durability, increased number of acidic sited and improve the photocatalytic effect of the nanocomposite.

The inventors of the present invention surprisingly found that it is essential to dry the aqueous phase directly out of the porous solid structure.

Dry the aqueous phase directly out of the porous solid structure may preferably not involve use of further chemicals, like acetone, CO₂, freon, or the like.

Traditionally, wet aerogels, e.g. hydrogels, are dried using supercritical drying, however, as mentioned herein drying using supercritical drying may be undesirable.

During supercritical drying the fluids in a liquid body crosses the boundary from liquid to gas, the liquid changes into gas at a finite rate, while the amount of liquid decreases.

When this happens, the surface tension in the liquid body pulls against any solid structures the liquid might be in contact with. Delicate structures such as silica gels, as provided by the present invention tend to brake by this surface tension as the liquid-gas-solid junction moves.

To avoid this, the fluids are be kept at a stage without crossing any phase boundaries, but instead passing through a supercritical region, where the distinction between gas and liquid ceases to apply. Densities of the liquid phase and vapor phase become equal at critical point of drying.

Generally, fluids considered suitable for supercritical drying include carbon dioxide and freon. Nitrous oxide may be used too, since nitrous oxide may have similar physical behaviour as carbon dioxide but is a powerful oxidizer at the supercritical state.

However, drying of supercritical water present in aerogels, or in aerogel-like structures like the porous solid structure of the present invention, may be undesirable due to increased risk of heat damage to the solid structure at the critical point temperature and corrosiveness of water at high temperatures and pressures.

Traditionally this is in most such processes solved by first using acetone to wash away all water from the gel structure, exploiting the complete miscibility of these two fluids. The acetone is then washed away with high pressure liquid carbon dioxide, the industry standard now that freon is unavailable. The liquid carbon dioxide is then heated until its temperature goes beyond the critical point, at which time the pressure can be gradually released, allowing the gas to escape and leaving a dried product.

Hence, supercritical drying of the aqueous phase in the porous solid structure of the present invention may be undesirable because it involves a very complex process with a lot of process steps which may influence the costs, added waste, use of additional process equipment's etc. which may be undesirable.

In an embodiment of the present invention the drying (step (iv)) does not involve supercritical drying of the aqueous phase in the porous solid structure.

In a further embodiment of the present invention the aqueous phase in the porous solid structure may be dried directly from the hydrogel.

The drying of the hydrogel at a temperature and pressure combination that avoids boiling of the aqueous phase, providing the nanocomposite, may preferably be evaporation of the aqueous phase under conditions that does not impose destructive boiling to the nanocomposite structure.

In an embodiment of the present invention gentle boiling may be allowed. Preferably, intensive boiling should be avoided to avoid destructive boiling to the nanocomposite structure.

The water of the hydrogel may be removed from the porous solid structure by a specific combination of temperature and pressure and therefore avoiding supercritical fluids.

In an embodiment of the present invention the aqueous phase in the aerogel may be dried at a temperature less than 105° C. at atmospheric pressure (about 1 bar pressure), e.g. at a temperature of 100° C. or less, such as at a temperature of 95° C. or less, e.g. at a temperature of 90° C. or less.

In yet an embodiment of the present invention the aqueous phase in the aerogel may be dried at a temperature above 10° C. at atmospheric pressure (about 1 bar pressure), such as at a temperature of 25° C. or higher, e.g. at a temperature of 50° C. or higher, such as at a temperature of 75° C. or higher, e.g. at a temperature of 85° C. or higher.

The aqueous phase in the aerogel may be dried at a temperature in the range of 10-105° C. at atmospheric pressure (about 1 bar pressure), e.g. at a temterature in the range of 20-99° C., such as at a temperature in the range of 25-96° C., e.g. at a temperature in the range of 50-94° C., such as at a temperature in the range of 75-92° C., e.g. at a temperature in the range of 85-90° C.

The skilled person would easily change temperature and pressure in relation to each other to provide a drying process where the aqueous phase in the porous solid structure is removed, or substantially removed, without boiling, or without intensive boiling, of aqueous phase in the porous solid structure.

In an embodiment of the present invention, the drying in step (iv) may be continued until a constant, or substantial constant, weigh of the aerogel may be obtained, or a substantial constant weight of the aerogel may be obtained.

The preparation of the homogenous mixture comprising the silica compound with one or more metal compounds in an aqueous solution may be provided by:
a) providing an aqueous solution comprising the metal compound, e.g. by providing particles of the one or more metal compounds in water, or initially dissolving a salt of the one or more metal compounds in water (preferably mixing the one or more metal salt in the water for 2-60 minutes, such as for 5-45 minutes, e.g. for 10-30 minutes, such as for about 15 minutes);
b) adding a silica compound to the water comprising the one or more metal oxide particles or the one or more dissolved metal salt; and
c) homogenize the mixture comprising the silica compound with one or more metal compounds (metal oxide particles or dissolved metal oxide salt). Preferably, homogenization of the mixture comprising the one or more metal compound and the silica compound may be continued for a period of 10-500 minutes, such as for a period of 20-350 minutes, e.g. for a period of 30-240 minutes, such as for 60-180 minutes, e.g. for 90-150 minutes, such as for about 120 minutes), providing the homogenous mixture comprising the silica compound and the one or more metal compound.

The homogenous mixture may, preferably before adjusting the pH, be a mixture of substances (silica compound and one or more metal compound) blended so thoroughly to provide a substantially uniform composition of the compounds and where every sample of the homogenous mixture may show the substantially the same amounts of each compound.

In an embodiment of the present invention the homogenous mixture comprising silica compound and one or more metal compounds may be acidic, such as strongly acidic.

Preferably, the pH value of the homogenisation mixture during homogenisation (in step (i)) is below pH 5, such as below pH 4, e.g. below pH 3, such as below pH 2, e.g. below pH 1.5, such as below pH 1, such as in the range of pH 0-5, e.g. in the range of pH 0.5-4, such as in the range of pH 1-3, e.g. in the range of pH 1.5-2.

In an embodiment of the present invention the mixing of the silica compound with the one or more metal compounds providing the homogenous mixture (step (i)) may be continued for a period of 10-500 minutes, such as for a period of 20-350 minutes, e.g. for a period of 30-240 minutes, such as for 60-180 minutes, e.g. for 90-150 minutes, such as for about 120 minutes, preferably under acidic conditions.

The homogenisation of the mixture comprising the one or more metal compound and the silica compound may be performed by heavy mixing.

Heavy mixing may involve mechanical mixing or magnetic stirring. Preferably, heavy mixing may provide an energy input by a mixing speed of 50 rpm or more, such as at least 100 rpm or more, e.g. at least 150 rpm, such as at least 200 rpm or more, e.g. at least 250 rpm, such as at least 300 rpm or more, e.g. at least 350 rpm, such as at least 400 rpm or more, e.g. at least 450 rpm, such as at least 500 rpm or more, e.g. at least 600 rpm.

The silica compound and/or the metal compounds provided in the present invention may be provided by a process commonly known to the skilled person.

The one or more metal compounds provided in step a) may be provided as a salt, preferably as an inorganic salt.

In an embodiment of the present invention at least one of the one or more metal compounds may comprise self-dispersing properties.

Self-dispersing properties may relate to improved properties in mixing and dispersing of at least one of the one or more metal compounds in a continuous phase of the silica.

In an embodiment of the present invention the silica compound may have a mean particle size d50 in the range of 3-20 nm, such as in the range of 4-15 nm, e.g. in the range of 5-nm, such as in the range of 6-8 nm, e.g. about 7 nm.

I yet an embodiment of the present invention the silica compound may have a specific surface area, a BET surface area, in the range of 200-600 m²/g, such as in the range of 250-500 m²/g, e.g. in the range of 300-450 m²/g, such as in the range of 350-425 m²/g, e.g. in the range of 380-400 m²/g.

The silica compound may be added to the water comprising the one or more metal compound resulting in a content of the silica compound of 50-90 wt %, such as 60-85 wt %, e.g. 70-80 wt %, such as about 75 wt %.

In an embodiment of the present invention the porous solid structure comprises 50-90 wt % silica compound, such as in the range of 60-85 wt % silica compound, e.g. in the range of 70-80 wt % silica compound, e.g. in the range of 73-77 wt % silica compound, e.g. about 75 wt % silica compound.

The amount silica added to the water comprising the one or more metal compound may be sufficient to allow the silica compound just to absorb all, or substantially all the water (including the one or more metal compound).

The amount of silica compound added to the water comprising the one or more metal compound may constitute more than 60 vol % of the volume of the water comprising the one or more dissolved metal and silica compound, such as more than 70 vol %, e.g. more than 80 vol %, such as more than 90 vol %, e.g. more than 95 vol %, such as more than 96 vol %, e.g. more than 98 vol %. This high volume of silika/metal compound may be provided by the nanocomposite material of the present invention because the silika/metal compound may additionally comprise large amounts of the water inside the silika/metal compound material.

In step (ii) of the process according to the present invention the pH of the homogenous mixture in favour of the reaction conditions forming the porous solid structure.

Preferably the pH may be adjusted to a pH of the homogenous mixture in the range of pH 6-10, such as a pH in the range of pH 7-9, e.g. a pH in the range of pH 7-8, such as a pH in the range of pH 8-9.

Preferably the change in pH is provided by the addition of a base, preferably, the base is ammonia.

In step (iii) of the process according to the present invention gelation of the silica compound and the one or more metal compounds present in the homogenous mixture may be provided by change in the pH of the homogenous mixture.

Preferably, the change in pH of the homogenous mixture comprising the silica compound and the one or more metal compounds may be performed by slowly addition of an acid or a base keeping the pH of the aqueous silica sol within the desired pH-range.

The step of gelation (step iii) of the homogenous mixture comprising the silica compound and the one or more metal compounds, may be allowed to proceed for a period of 10-500 minutes, such as for a period of 20-350 minutes, e.g. for a period of 30-240 minutes, such as for 60-180 minutes, e.g. for 90-150 minutes, such as for about 120 minutes.

Preferably, the gelation process resulting in the hydrogel comprising the aqueous phase and the porous solid structure may be continued for a period of 10-500 minutes, such as for a period of 20-350 minutes, e.g. for a period of 30-240 minutes, such as for 60-180 minutes, e.g. for 90-150 minutes, such as for about 120 minutes.

In an embodiment of the present invention the hydrogel formed during gelation in step (iii) may be comprise an aqueous phase and an porous solid structure.

During the step of gelation (step (iii)) the homogenous mixture comprising the silica compound and the one or more metal compounds, and/or the hydrogel provided in step (iii), may be subjected to heavy mixing to provide sufficient mixing of acid/base and the homogenous mixture and providing a homogeneous hydrogel comprising an aqueous phase and a porous solid structure having a porous monolithic inorganic gel structure comprising a silica compound and one or more metal compounds.

In an embodiment of the present invention, heavy mixing may involve mechanical mixing or magnetic stirring. Preferably, heavy mixing may provide an energy input obtained from a mixing speed of 50 rpm or more, such as at least 100 rpm or more, e.g. at least 150 rpm, such as at least 200 rpm or more, e.g. at least 250 rpm, such as at least 300 rpm or more, e.g. at least 350 rpm, such as at least 400 rpm or more, e.g. at least 450 rpm, such as at least 500 rpm or more, e.g. at least 600 rpm.

Furthermore, after mixing the pH adjusted hydrogel may be allowed to settle.

In an embodiment of the present invention the hydrogel may be allowed to settle for at least 1 hour, such as for at least 2 hours, e.g. for at least 3 hours, such as for at least 4 hours, e.g. for at least 5 hours, such as for at least 7 hours, e.g. for at least 10 hours, such as for at least 15 hours.

In a further embodiment of the present invention the hydrogel may be allowed to settle simultaneously, or substantially simultaneously, with a step of removing salts and/or ions before drying the hydrogel in step (iv).

The substantially simultaneously settlement of the hydrogel and removal of salts and/or ions may include at least 20% of the time for settling the hydrogel may be performed together with the removal of salts and/or ions, such as at least 30% of the time, e.g. at least 40% of the time, such as at least 50% of the time, e.g. at least 60% of the time, such as at least 70% of the time, e.g. at least 80% of the time, such as at least 90% of the time, e.g. at least 95% of the time.

The process according to the present invention may be a process for providing the nanocomposite according to the present invention.

In an embodiment of the present invention the hydrogel may be subjected to a step of removing salts and/or ions before drying the hydrogel in step (iv).

Preferably, the step of removing salts and/or ions may include dialysis.

Dialysis of the hydrogel may result in a dialysed hydrogel.

Dialysis may be continued for at least 4 hours, such as for at least 8 hours, e.g. at least 12 hours, such as for at least 16 hours, e.g. at least 20 hours, such as for at least 24 hours, e.g. at least 28 hours.

Preferably, dialyses may be performed at elevated temperatures above room temperature. Elevated temperatures may be temperatures 30° C. or above, such as temperatures 35° C. or above, e.g. temperatures of 40° C. or above, preferably about 40° C.

The dialysis may preferably be performed with water, in particular demineralised water. The water may be changed with fresh water for at least every 4 hours.

After dialysis, the dialysed hydrogel may be collected by separating the dialysed hydrogel from the water.

Separation of the dialysed hydrogel from the water may be performed by sedimentation, decanting, and/or centrifugation.

To obtain proper separation of the dialysed hydrogel from the water by sedimentation the dialysed hydrogel may be allowed to sediment for at least 12 hours, such as for at least 24 hours, e.g. for at least 2 days, such as for at least 5 days, e.g. for at least 10 days, such as for at least 15 days, e.g. for at least 20 days, such as for at least 25 days, e.g. for at least 30 days, such as for at least 35 days.

The separated hydrogel may be subjected to a drying process as described in step (iv) providing the nanocomposite.

The drying of the hydrogel may be performed at a temperature and pressure combination that avoids boiling of the aqueous phase, providing the nanocomposite. The boiling may preferably involve evaporation of the aqueous phase under conditions that does not impose destructive boiling to the nanocomposite structure.

In an embodiment of the present invention gentle boiling may be allowed. Preferably, intensive boiling should be avoided to avoid destructive boiling to the nanocomposite structure.

The skilled person would easily change temperature and pressure in relation to each other to provide a drying process where the aqueous phase in the porous solid structure may be removed, or substantially removed, without boiling, or without intensive boiling, of aqueous phase in the porous solid structure.

Following the drying process, the dried nanocomposite may be subjected to comminution, e.g. by milling the nanocomposite, providing a fine powder comprising the nanocomposite.

In an embodiment of the present invention the fine powder comprising the nanocomposite may have a specified mean particle size (d50) according to the application of the nanocomposite. The skilled person would know the preferred specified mean particle size (d50) as well as means for milling the nanocomposite to the specified mean particle size (d50).

In an embodiment of the present invention the nanocomposite may be subjected to a calcination process providing a calcined nanocomposite.

In an embodiment of the present invention the nanocomposite may be a calcined nanocomposite.

Preferably, the calcination process may be performed at a calcination temperature in the range of 400-1000° C., such as in the range of 500-900° C.

The calcination process may be performed for a period of 30 minutes to 24 hours, such as for a period of 1-12 hours, e.g. for a period of 2-6 hours, e.g. for about 3 hours.

The period of the calcination process may be determined as the calcination time at the specified calcination temperature.

After calcination the nanocomposite may comprise an amorphous structure and/or a crystalline structure.

The nanocomposite may comprise an amorphous structure and a crystalline structure since the change from the amorphous structure to the crystalline structure may be provided gradually depending on calcination time and calcination temperature.

The calcination process may result in a change in the nanocomposite from an amorphous structure to a crystalline structure of the silica compound.

The amorphous structure may be resembling liquids in that they do not have an ordered structure, an orderly arrangement of atoms or ions in a three-dimensional structure. This amorphous structure may not have a sharp melting point and the solid to liquid transformation occurs over a range of temperatures.

The crystalline structure/crystalline anatase structure may have distinctive internal structures that in turn lead to distinctive flat surfaces, or faces. The faces intersect at angles that are characteristic of the substance. When exposed to x-rays, each structure also produces a distinctive pattern that can be used to identify the material.

The crystalline structure of the calcined nanocomposite may provide a solid structure which has and increased electrical conductivity, increased thermal conductivity, increased mechanical strength, increased refractive index, more stable, increased durability, and/or increased rigidity compared to the amorphous structure.

The choice of structure of the nanocomposite (being amorphous structure or crystalline structure) may depend on the intended use of the nanocomposite.

In an embodiment of the present invention the nanocomposite may be provided in a crystalline structure, or mainly in a crystalline structure, which may be a partly crystalline structure or a mixed crystalline structure.

The nanocomposite comprising the dried nanocomposite or the calcined nanocomposite, preferably, the calcined nanocomposite, may be coated with any suitable agent, such as a couplings agent depending on the intended use of the nanocomposite.

The nanocomposite obtained by the process according to the present invention may be formulated into a nanoparticulate material.

The nanocomposite and the nanoparticulate material according to the present invention may find suitable application in various fields.

A preferred embodiment of the present invention may relate to the use of an nanocomposite according to the present invention, as a filler in paint (preferably, in outdoor paints or marine paints; more preferably in outdoor paints); coating of a medical device; or as a dental filling.

An embodiment of the present invention relates to a paint comprising an nanocomposite according to the present invention.

Paints may be applied on the surfaces of various materials like timber, metals and plastered surfaces as a protective layer and at the same time to get pleasant appearance. Paints may be applied to dry or wet surfaces. Paints may be applied in liquid form and after a period the volatile constituent may be evaporated and the resulting hardened coating acts as a protective layer of the surface.

In the present context the term “wet surfaces” may relate to surfaces that regularly are subjected to water (fresh water, rainwater and/or sea water).

In an embodiment of the present invention the paint may be a paint intended for wet surfaces, preferably, outdoor paints or marine paints; more preferably in outdoor paints.

In addition to the aerogel the paint according to the present invention comprises a paint base, a paint vehicle, a paint pigment, a paint dryer and/or a paint thinner.

An embodiment of the present invention relates to a medical device comprising a nanocomposite according to the present invention.

An embodiment of the present invention relates to a dental filler comprising a nanocomposite according to the present invention.

A nanocomposite according to the present invention when used in various applications (such as a filler in paint (preferably, in outdoor paints or marine paints; more preferably in outdoor paints); e.g. as a coating of a medical device; or as a dental filling) may include a TiO₂/SiO₂ porous solid structure (silica titanium dioxide porous solid structure), or a ZrO₂/SiO₂ porous solid structure (silica zirconium dioxide porous solid structure), or a TiO₂/ZrO₂/SiO₂ porous solid structure (silica titanium dioxide zirconium dioxide porous solid structure), as the one or more metal compounds and the silica compound.

The ratio between TiO₂/SiO₂ may be in the range of 10-50:50-90 (preferably 25:75) and the ratio between ZrO₂/SiO₂ may be in the range of 10-50:50-90 (preferably 25:75). In the TiO₂/ZrO₂/SiO₂ porous solid structure the ratio may be 10-40:10-40:50-90 (preferably 10:25:65)

The porous solid structure according to the present invention may comprise 60-80% (w/w), such as 70-78% (w/w) e.g. about 75% (w/w) silica and 20-40% (w/w), e.g. 23-30% (w/w), e.g. about 25% zirconia. Preferably, the porous solid structure comprising 60-80% (w/w), such as 70-78% (w/w) e.g. about 75% (w/w) silica and 20-40% (w/w), e.g. 23-30% (w/w), e.g. about 25% zirconia may comprise a surface area (a BET surface area) in the range of 150-600 m²/g, such as in the range of 200-400 m²/g, e.g. in the range of 300-350 m²/g, such as about 335 m²/g.

The porous solid structure according to the present invention may comprise 50-75% (w/w), such as 60-70% (w/w) e.g. about 65% (w/w) silica; 20-40% (w/w), e.g. 23-30% (w/w), e.g. about 25% zirconia; and 5-15% (w/w), e.g. 8-12% (w/w), e.g. about 10% titania dioxide (preferably anatas nanoparticles). Preferably, the porous solid structure comprising 50-75% (w/w), such as 60-70% (w/w) e.g. about 65% (w/w) silica; 20-40% (w/w), e.g. 23-30% (w/w), e.g. about 25% zirconia; and 5-15% (w/w), e.g. 8-12% (w/w), e.g. about 10% titania dioxide (preferably anatas nanoparticles) may comprise a surface area (a BET surface area) in the range of 150-600 m²/g, such as in the range of 200-400 m²/g, e.g. in the range of 300-350 m²/g, such as about 315 m²/g.

The porous solid structure according to the present invention may comprise 50-75% (w/w), such as 60-70% (w/w) e.g. about 68% (w/w) silica; 20-40% (w/w), e.g. 22-30% (w/w), e.g. about 23% zirconia; and 5-15% (w/w), e.g. 8-12% (w/w), e.g. about 9% titania dioxide (preferably anatas nanoparticles). Preferably, the porous solid structure comprising 50-75% (w/w), such as 60-70% (w/w) e.g. about 68% (w/w) silica; 20-40% (w/w), e.g. 22-30% (w/w), e.g. about 23% zirconia; and 5-15% (w/w), e.g. 8-12% (w/w), e.g. about 9% titania dioxide (preferably anatas nanoparticles) may comprise a surface area (a BET surface area) in the range of 150-600 m²/g, such as in the range of 200-400 m²/g, e.g. in the range of 300-350 m²/g, such as about 323 m²/g.

The porous solid structure according to the present invention may comprise 60-80% (w/w), such as 70-78% (w/w) e.g. about 75% (w/w) silica and 20-40% (w/w), e.g. 23-30% (w/w), e.g. about 25% alumina. Preferably, the porous solid structure comprising 60-80% (w/w), such as 70-78% (w/w) e.g. about 75% (w/w) silica and 20-40% (w/w), e.g. 23-30% (w/w), e.g. about 25% alumina may comprise a surface area (a BET surface area) in the range of 75-400 m²/g, such as in the range of 100-300 m²/g, e.g. in the range of 125-200 m²/g, such as about 164 m²/g.

In an embodiment of the present invention the nanocomposite may comprise an internal porous volume and wherein the internal porous volume is filled with an internal material.

The internal material may be a gas, a liquid material or a solid material. When the internal material is a solid the solid material may be introduced as a liquid which is subsequently solidified inside the internal porous volume.

The nanocomposite be provided with one or more coatings. Preferably, the coating may be a coupling agent. Preferably, the couplings agent may be coupled to the nanocomposite by a chemical coupling to the surface of the porous solid structure, such as the TiO₂/SiO₂ porous solid structure and/or a ZrO₂/SiO₂ porous solid structure.

Due to the large surface area of the nanocomposite and the porous solid structure according to the present invention a range of silane couplings agents may be used.

Silane couplings agents may preferably be reactive silanes with a methacrylate ending, such as 3-(Trimethoxysilyl) propyl methacrylate, 3-(Triethoxysilyl) propyl methacrylate. These coupling agents makes the nanocomposite according to the present invention compatible with common dental acrylate resins, but also contribute to increase important material properties, as for example flexural strength, wear ability and gloss retention, in the hardened dental fillings. Without being bound by theory, the inventors of the present invention trust that these effects may be assigned to the formation of strong chemical bonding, through the coupling agent, between the surface of the porous solid structure and the acrylic resin matrix.

Another relevant coupling agent may be (3-Glycidoxypropyl) trimethoxysilane, or similar compounds, which beside the reactive silane end have an glycidyl in the other end. It makes it very useful in the same manner as before, but in connection with epoxy coatings instead of acrylic resins.

Aminosilanes could also be utilized in order to let the amine end react with epoxide groups from the coating system.

The nanocomposite according to the present invention may also be treated to adjust the level of water uptake. This may be done by reacting the surface of the porous solid structure with a range of different reactive silanes, e.g. methyltrimethoxy silane or the like. The methyl group introduced by such reactive silane may change the surface energy of the porous solid structure in such a way that the coating or resin mixture itself will perform more hydrophobic, which again may lead to an overall lower degree of water uptake when exposed to an environment with high humidity or directly in water.

It should be noted that embodiments and features described in the context of one of the aspects of the present invention also apply to the other aspects of the invention.

All patent and non-patent references cited in the present application, are hereby incorporated by reference in their entirety.

The invention may be further described in the following non-limiting examples.

### EXAMPLES

### Example 1: Synthesis of the Nanocomposite, Purification and Drying

72,5 g Zirconium (IV) oxynitrate hydrate (Technical grade from SIGMA-Aldrich Co.) was dissolved in 2 L demineralized water.

80,1 g pyrogenic silicate (S5130 from SIGMA-Aldrich Co.) was dispersed in the zirconium solution under heavy mixing at 400 rpm. The pH value of solution comprising pyrogenic silicate and the zirconium solution (zirconium (IV) oxynitrate hydrate) was found to be pH 1.

Mixing is done in 3 hours and under thoroughly mixing and ammonia was added dropwise for pH adjustment (1410 g of 0,365M ammonia solution was used). pH was adjusted to 8-9.

After slow mixing overnight for allowing reaction to take place, pH was found to be 6-7.

Staying without mixing allow the hydrogel formed to settle and after that 0,9 L liquid was decanted off.

The hydrogel was placed in dialysis tubes and dialyzed against demineralized at 45° C. During dialysis the gel settled further and filled approximately half part of the volume of the dialysis tube. The dialysis took place until the mother liqueur separated above the hydrogel in the tubes was measured to contain 88 ppm of salt. The mother liqueur was then decanted of and the dialysed hydrogel collected in a glass bowl.

The dialysed hydrogel was dried in an oven at 90° C. for 92 hours forming the nanocomposite comprising an porous solid structure having a porous monolithic inorganic gel structure. 103,3 g dried gel (nanocomposite) was collected for calcination.

### Example 2: Comminution and Calcination of the Nanocomposite from Example 1

Some pieces of the nanocomposite from example 1 was milled down to fine powder for 90 seconds in an OBH mill. 20,7 g of the obtained material was calcined at 500° C. for 3 hours giving 19 g of the final nanocomposite.

### Example 3: Synthesis of the Nanocomposite from an Oxychloride Precursor, Purification and Drying

140,0 g Zirconyl chloride octahydrate (99% purity from HEALTH BIOCHEM TECHNOLOGY CO.,LTD) was dissolved in 4,0 L demineralized water.

175 g hydrophilic pyrogenic silica (HDK® T40 from Wacker Chemie AG) was dispersed in the solution. Mixing was performed at 400 rpm for 3 hours. The pH value of solution comprising pyrogenic silicate and the zirconium solution (zirconium (IV) oxynitrate hydrate) was found to be pH 1-2.

Under thoroughly mixing pH was adjusted by dropwise addition of 2.90 kg of 0,365M ammonia solution. pH was approximately 9. After slow mixing overnight for allowing reaction to take place, pH was found to be approximately 8.

The hydrogel formed was transferred into dialysis tubes and dialyzed against demineralized at 45° C. During dialysis the hydrogel settled further and filled approximately half part of the volume of the dialysis tube. The dialysis took place until the mother liqueur separated above the hydrogel in the tubes was measured to contain 73 ppm of salt. The mother liqueur was then decanted off and 3.11 kg dialysed hydrogel was collected in a glass bowl.

The dialysed hydrogel was dried in an oven at 90° C. for 44 hours resulting in a nanocomposite. 219,2 g nanocomposite was collected for calcination providing the porous solid structure having a porous monolithic inorganic gel structure.

### Example 4: Comminution and Calcination of the Nanocomposite from Example 3

Some pieces of the nanocomposite from example 3 was milled down to fine powder for 2 times 45 seconds in an OBH mill. 217,77 g of the obtained material was calcined at 500° C. for 3 hours giving 198,53 g of the final nanocomposite.

### Example 5: Synthesis of the Nanocomposite with Incorporated TiO₂ Nanoparticles, Purification and Drying

7,01 g Zirconium (IV) oxychloride hydrate was dissolved in a transparent dispersion of 1,01 g TiO₂ 5 nm anatase nanoparticles (JR05 from Xuancheng Jingrui New Material Co., ltd.) in 0,2 L demineralized water.

7,40 g hydrophilic pyrogenic silica (HDK® T40 from Wacker Chemie AG) was further dispersed in the mixture. Mixing was done at 400 rpm for 3 hours. The pH value of solution comprising pyrogenic silicate and the zirconium solution (zirconium (IV) oxynitrate hydrate and TiO₂) was found to be pH 1.

Under thoroughly mixing pH was adjusted by dropwise addition of 150,3 g of 0,365M ammonia solution. pH was then 8-9. After slow mixing overnight for allowing reaction to take place, pH was found to be 7.

The hydrogel formed was transferred to dialysis tubes and dialysed against demineralized at 20° C. During dialysis the hydrogel settled further and filled approximately half part of the volume of the dialysis tube. The dialysis took place until the mother liqueur separated above the hydrogel in the tubes was measured to contain 11 ppm of salt. This mother liqueur was then decanted off and 240 g dialysed hydrogel was collected in a glass bowl.

The dialysed hydrogel was dried in an oven at 90° C. for 24 hours resulting in a nanocomposite. 11,13 g nanocomposite was collected for calcination comprising a porous solid structure having a porous monolithic inorganic gel structure.

### Example 6: Comminution and Calcination of the Dried Mixed Oxide Gel from Example 5

Some pieces of the nanocomposite from example 5 was milled down to fine powder for 2 times 45 seconds in an OBH mill. 10,78 g of the obtained material was calcined at 500° C. for 3 hours giving 9,99 g of the final nanocomposite.

Example 7: Test of conservation of the photocatalytic effect of anatase nanoparticle in the material prepared in example 6 relative to the nanoparticle in the material prepared in example 4.

Nanocomposites prepared as described in examples 4 and 6 (calculated to have a TiO₂ content of 10 weight-%) was placed under a solution of Rhodamine B and exposed to UV light at 365 nm in interval of 3 minutes under stirring.

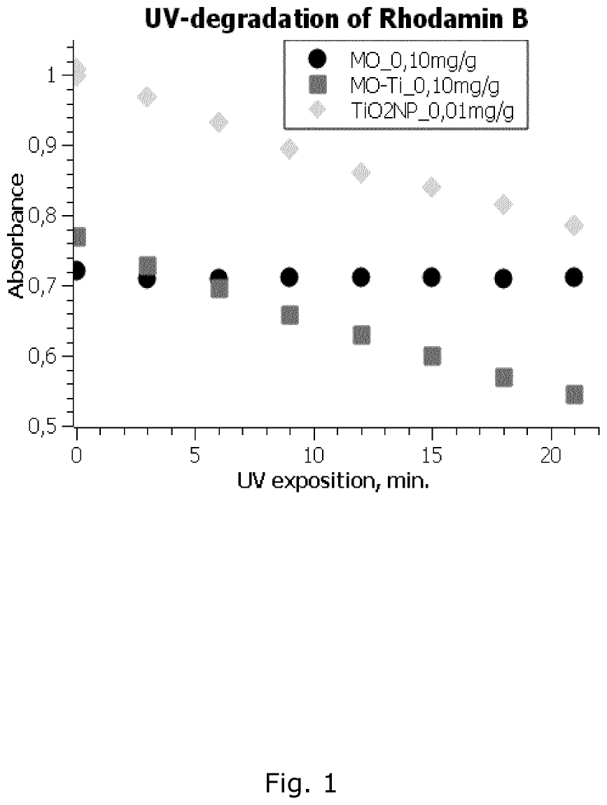

The photocatalyzed degradation of Rhodamine B was followed spectrophotometrically and compared to the degradation obtained from a dispersion of the equal amount of the free dispersed TiO₂ nanoparticles, see FIG. 1.

Black circular spots represent the effect of the nanocomposite prepared in example 4 comprising SiO₂/ZrO₂ and show no measurable photoactivity in a concentration of 0.10 mg/g Rhodamine B solution.

Red squares represent the effect of the nanocomposite prepared in example 6 with 10 weight-% of TiO₂ anatase 5 nm nanoparticles included into the porous solid structure (SiO₂/ZrO₂/TiO₂) and show similar rate of photoactivity in a concentration of 0.10 mg/g Rhodamine B solution, compared to the green rhomb's representing equal concentration of TiO₂ anatase 5 nm nanoparticles, but in free dispersion.

1. A process for providing a nanocomposite, the process comprising the steps of:
(i) Mixing a silica compound with one or more metal compounds in an aqueous solution providing a homogenous mixture;
(ii) Adjusting the pH of the homogenous mixture providing a pH-adjusted homogenous mixture;
(iii) Allowing pH-adjusted homogenous mixture silica to undergo a gelation process resulting in a hydrogel comprising an aqueous phase and an porous solid structure;
(iv) Drying the hydrogel at a temperature and pressure combination that avoids boiling of the aqueous phase, providing the nanocomposite;
wherein the nanocomposite provided may have a porous monolithic inorganic gel structure comprising a silica compound and one or more metal compounds.

2. The process according to claim 1, wherein the one or more metal compound is one or more metal oxide compounds or one or more metal hydroxide compounds.

3. The process according to claim 1, wherein the metal compound comprises a divalent metal ion, a trivalent metal ion, a tetravalent metal ion.

4. The process according to claim 1, wherein the mixing of the silica compound with the one or more metal compounds providing the homogenous mixture (step (i)) is continued for a period of 10-500 minutes, preferably under acidic conditions.

5. The process according to claim 1, wherein the adjustment of the pH of the homogeneous mixture in step (ii) is to an alkaline pH or to a pH-value in the range of pH 6-10.

6. The process according to claim 1, wherein the gelation process resulting in the hydrogel comprising the aqueous phase and the porous solid structure is continued for a period of 10-500 minutes.

7. The process according to claim 1, wherein the aqueous phase in the hydrogel may be dried at a temperature less than 100° C. at atmospheric pressure (at about 1 bar pressure), such as at a temperature of 95° C. or less, e.g. at a temperature of 90° C. or less.

8. The process according to claim 1, wherein the hydrogel is subjected to a step of removing salts and/or ions before drying the hydrogel in step (iv).

9. The process according to claim 1, wherein the step of removing salts and/or ions includes dialysis.

10. The process according to claim 1, wherein the nanocomposite comprises an internal porous volume and wherein the internal porous volume is in the range of 40-90%.

11. A nanocomposite comprising an porous solid structure having a porous monolithic inorganic gel structure comprising a silica compound and one or more metal compounds obtainable by the method according to claim 1.

12. A nanocomposite comprising an porous solid structure having a porous monolithic inorganic gel structure comprising a silica compound and one or more metal compounds, wherein the porous solid structure comprises an internal porous volume in the range of 40-90%, such as in the range of 50-80%, e.g. in the range of 60-75%, such as about 70%.

13. The nanocomposite according to claim 11, wherein the porous solid structure comprises a surface area in the range of 10-1500 m²/g, such as in the range of 20-1200 m²/g, e.g. in the range of 25-1000 m²/g, such as in the range of 30-800 m²/g, e.g. in the range of 40-600 m²/g; such as in the range of 50-300 m²/g, e.g. in the range of 75-250 m²/g; such as in the range of 100-200 m²/g.

14. The nanocomposite according to claim 11, wherein the porous solid structure comprises 50-90 wt % silica compound, such as in the range of 60-85 wt % silica compound, e.g. in the range of 70-80 wt % silica compound, e.g. in the range of 73-77 wt % silica compound, e.g. about 75 wt % silica compound.

15. Use of a nanocomposite according to claim 1 as a filler in paint (preferably, in outdoor paints or marine paints; more preferably in outdoor paints); as a coating of a medical device; or as a dental filling.
